(19) United States
(12) Patent Application Publication
(10) Pub. No.: US 2026/0363911 A1
(43) Pub. Date: Nov. 19, 2026

(54) AN IMPLANTABLE OCULAR DRAINAGE DEVICE FOR CONTROLLING INTRAOCULAR PRESSURE

(71) Applicants: UNIVERSITEIT MAASTRICHT, MAASTRICHT (NL); ACADEMISCH ZIEKENHUIS MAASTRICHT, MAASTRICHT (NL); TECHNISCHE UNIVERSITEIT EINDHOVEN, EINDHOVEN (NL)

(72) Inventors: Christian Johannes Franciscus BERTENS, MAASTRICHT (NL); Helena Jacqueline Maria BECKERS, MAASTRICHT (NL); Jacob Marinus Jan DEN TOONDER, EINDHOVEN (NL); Rosanne Francisca VAN DE WIJDEVEN, EINDHOVEN (NL); Leonard PINCHUK, MIAMI, FL (US)

(21) Appl. No.: 19/154,357

(22) PCT Filed: Feb. 9, 2024
(86) PCT No.: PCT/EP2024/053354

§ 371 Date: Aug. 6, 2025

Related U.S. Application Data
(60) Provisional application No. 63/444,781, filed on Feb. 10, 2023.

(30) Foreign Application Priority Data
Mar. 2, 2023 (EP) 23159573.7

(51) Int. Cl. A61F 9/007

(52) U.S. Cl. CPC A61F 9/00781; A61F 2250/0013

(57) ABSTRACT

The implantable ocular drainage device is configured for controlling intraocular pressure in the eye. The ocular implantable drainage device comprises a wall body surrounding in an endless manner a reservoir with a reservoir volume defined by means of the wall body, wherein the device further comprises at least one fluid inlet, at least one fluid outlet for filling the reservoir with fluid and at least one fluid flow channel providing fluid communication between the at least one fluid inlet and the at least one fluid outlet.

The invention relates to an implantable ocular drainage device for controlling intraocular pressure.

Glaucoma is an eye disease and is the leading cause of unpreventable/irreversible blindness worldwide. A rise in the intraocular pressure (IOP) is considered to be the major risk factor for glaucoma and is associated with an unbalance between the production and drainage of aqueous humor, due to an abnormal increase of resistance to aqueous humor outflow. Next to pharmacotherapy which in many cases is not sufficiently effective and causes serious side effects, surgical implants are available to reduce IOP by providing an alternative pathway through which fluid, i.e. aqueous humor, can effectively drain, thereby lowering IOP in the eye. Current glaucoma implants are for example long-tube glaucoma drainage devices which have a relatively high failure rate. Moreover, implanting a long-tube glaucoma drainage device is difficult, time consuming, and can only be done by highly specialized surgeons. In time, the implants often fail, mainly due to scarring or fibrosis of the bleb which consequently results in raised intraocular pressure.

Another example of an implantable ocular drainage device is disclosed in WO2022/111892. This known device comprises a magnetic micro-valve mechanism to control fluid flow such that the intraocular pressure in the eye can be maintained at a healthy value.

It is an object of the present invention to provide an improved implantable ocular drainage device.

This object is achieved with the implantable ocular drainage device as defined in claim 1.

The implantable ocular drainage device is configured for controlling intraocular pressure in the eye. The implantable ocular drainage device comprises a wall body surrounding in an endless manner a reservoir with a reservoir volume defined by means of the wall body, wherein the device further comprises at least one fluid inlet, at least one fluid outlet for filling the reservoir with fluid and at least one fluid flow channel providing fluid communication between the at least one fluid inlet and the at least one fluid outlet, wherein the wall body is provided with at least one overflow member configured to control a maximum volume of fluid in the reservoir, wherein the maximum volume of fluid in the reservoir is smaller than the reservoir volume.

The ocular implantable drainage device with the reservoir defined by the wall body of the device is designed to collect fluid (hereafter aqueous humor) in the reservoir, wherein after collecting a predetermined maximum volume of aqueous humor in the reservoir the aqueous humor leaves the device in a distributed manner by means of the overflow member of the wall body.

The overflow member may be configured to direct aqueous humor flow in a predetermined direction away from the device to obtain optimal drainage results. In addition, optimal bleb formation around the implanted ocular drainage device is achieved by the aqueous humor being pooled in the reservoir and the overflow member distributing the aqueous humor out of the device. The bleb is the final barrier for aqueous humor before resorption into the venous system or through the conjunctiva or lymphatics into the tear film. The size and cell composition of the bleb have a major impact on IOP. Poor bleb formation will results in failure of a glaucoma drainage device as a result of fibrosis and scarring of the filtering bleb. The design of the implanted ocular drainage device positively contributes to improved size and cell composition of the bleb, in that the design facilitates bleb-formation including an appropriately sized bleb that minimizes resistance to aqueous humor outflow, such that after implantation the improved ocular drainage device has a relatively low failure rate.

In addition, the design of the device which is mainly provided by the endless wall body facilitates implanting of the device, i.e., making implanting of the device relatively easy, in particular (stable) positioning of the device at the desired and most optimal location such that an effective drainage of aqueous humor can be obtained by means of the device.

In one aspect, the endless wall body, seen from above, defines a center providing a virtual origin used for defining a virtual x- and y-axis, wherein, seen from above, the position of the fluid inlet coincides with the y-axis and at least a portion of the at least one overflow member is provided in a wall body section of the wall body opposite to the fluid inlet with respect to the x-axis.

This configuration of the device having at least one posteriorly located overflow member, has the result that aqueous humor flow is distributed posteriorly, such that bleb formation is directed further onto the bulbus, which facilitates optimal bleb formation for drainage by minimizing fibrosis of the bleb. This position of the overflow member or members with respect to the fluid inlet and the at least upwards open reservoir provide in an implanted state of the device an environment around the device which results in excellent drainage results.

In one further aspect, the endless wall body, seen from above, defines a center providing a virtual origin used for defining a virtual x-and y-axis, wherein, seen from above, the position of the fluid inlet coincides with the y-axis and at least a portion of the at least one fluid outlet is provided in a wall body section of the wall body opposite to the fluid inlet with respect to the x-axis.

The posteriorly located fluid outlet for filling the reservoir in the implanted device maximizes tissue healing after implantation and drains fluid in a posterior location of the surgical site.

The implantable ocular drainage device may be made of a biocompatible, non-erodible polymer. Exemplary polymers include silicone rubber, polypropylene, polytetrafluoroethylene, and preferably poly(styrene-block-isobutylene-block-styrene). The device made of a biocompatible, non-erodible polymer can be implanted by means of a relatively easy surgical procedure, as the device does not need to be patched with (human donor) sclera or other material to prevent conjunctival erosion.

Further, the wall body may be ring-shaped or ellipse shaped. A ring-shaped device is well-suited for sitting firmly on a spherical surface, such as the eye-ball. Hence, ringlike shapes provide a stable positioning on the eye-ball which contributes in a relatively high success rate of implanted devices. The device may also comprise a curved-design adapted to the curvature of the eye. Such a curved-design also facilitates positioning of the device in the eye, but also reduces strain and/or deformation after implanting the device.

In addition, or alternatively, it is possible to include the magnetic micro-valve mechanism as disclosed in WO2022/111892 in the ocular drainage device of the present disclosure, wherein the device with the magnetic micro-valve mechanism is positioned in the reservoir volume, for example in the center, such that the device of this disclosure is mainly provided around the known device.

In one embodiment, the at least one overflow member is a notch in the wall body, in particular the top of the wall body. In this way, the wall body provides a reservoir volume defined by the height of the wall body, wherein the wall body has a lower height at the at least one notch, such that the maximum volume of fluid to be collected in the reservoir is smaller than the reservoir volume which is defined by the height of the wall body sections(s) without a notch.

Alternatively, or in addition, the overflow member may also be provided by means of at least one opening though the wall body under the top side of the wall body, such that above the maximum volume fluid in the reservoir fluid will exit the reservoir by means of the at least one opening.

The present invention will be explained in more detail below with reference to the appended figures showing exemplary embodiments, in which:

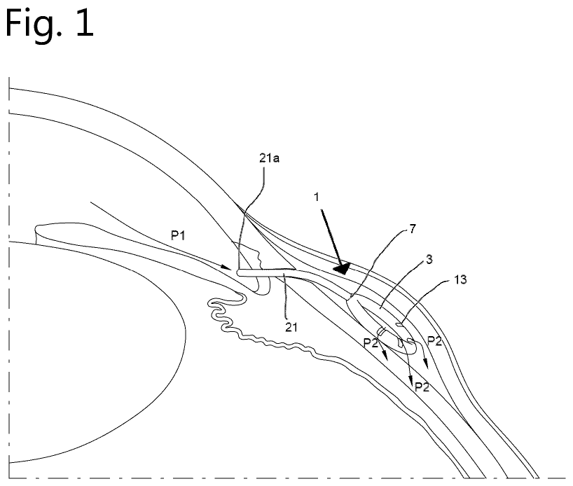

FIG. 1 shows a diagrammatic view of a first embodiment of the implantable ocular drainage device after implantation;

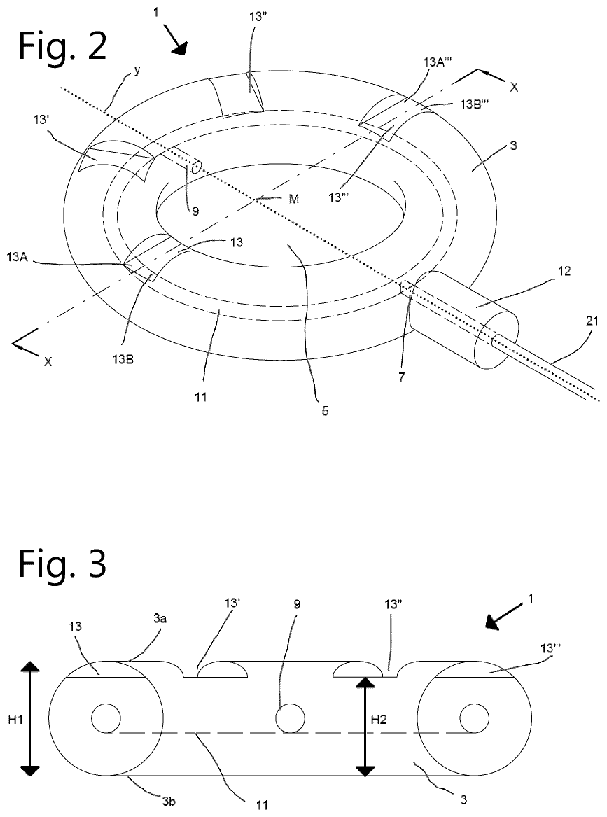

FIGS. 2 and 3 show a perspective view and a cross-sectional view of the implantable ocular drainage device shown in FIG. 1;

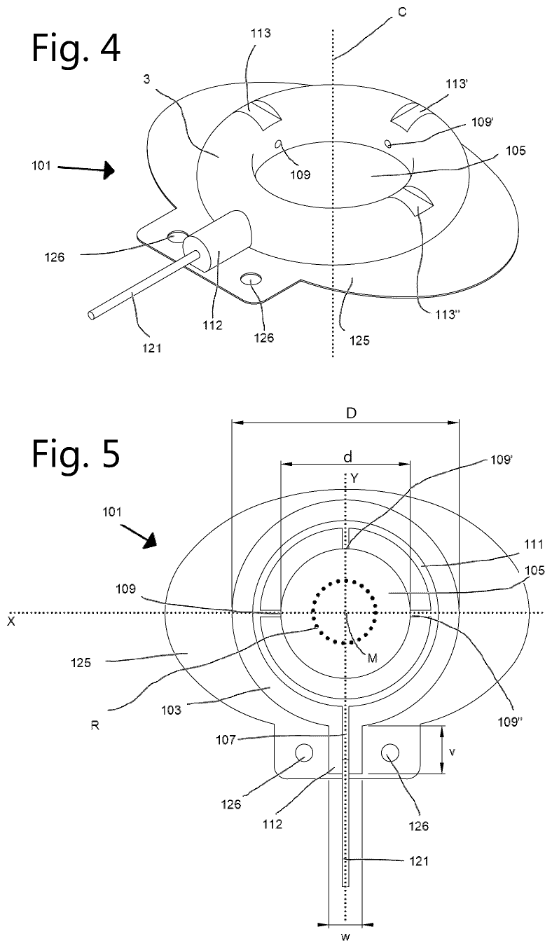

FIG. 4 shows a perspective view of a second embodiment of the implantable ocular drainage device;

FIG. 5 shows a diagrammatic top view of the implantable ocular drainage device shown in FIG. 4;

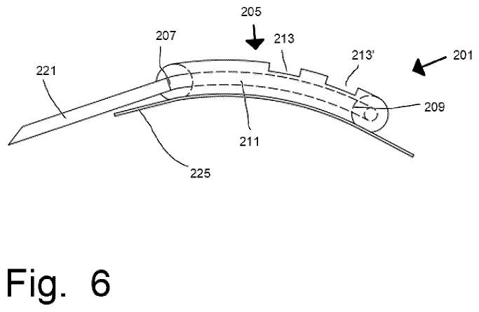

FIG. 6 shows a diagrammatic view of a third embodiment of the implantable ocular drainage device.

In the following description identical or corresponding parts have identical or corresponding reference numerals. Each feature disclosed with reference to a specific figure can also be combined with another feature disclosed in this disclosure, unless it is evident for a person skilled in the art that these features are incompatible. The FIGS. 1-6 show various embodiments of an implantable ocular drainage device 1; 101; 201 for controlling intraocular pressure. The ocular drainage device 1; 101; 201 comprises a wall body 3; 103; 203 surrounding in an endless manner a reservoir 5; 105; 205 with a reservoir volume defined by means of the wall body 3; 103; 203. The device 1; 101; 201 further comprises at least one fluid inlet 7; 107; 207, at least one fluid outlet 9; 109, 109′, 109″ 209 for filling the reservoir with fluid and at least one fluid flow channel 11; 111 211 providing fluid communication between the at least one fluid inlet and the at least one fluid outlet, wherein the wall body 3; 103; 203 is provided with at least one overflow member 13, 13′, 13″, 13″′; 113, 113′, 113″; 213, 213′ configured to control a maximum volume of fluid in the reservoir, wherein the maximum volume of fluid in the reservoir is smaller than the reservoir volume.

The device 1; 101; 201 further comprises a tube 21; 121; 221, wherein the tube is connected or connectable to the fluid inlet 7; 107; 207. The device 1; 101 has a tube connector 12; 112 to be connected to the wall body 3; 103 or tube connector 12; 112 is in one-piece with the wall body. The tube connector has dimensions in the x-y plane defined by v (1-2 mm) and w (0.5-1 mm) and a height corresponding to the height of the wall body 3; 103. FIG. 1 shows an implanted ocular drainage device 1, wherein the embodiments of the device 101; 201 shown in FIGS. 4-6 may be implanted in the same manner in the eye. FIG. 1 shows that the ring-shaped wall body 3 is designed to be positioned in the sub-conjunctival/sub-Tenon's space and an end 21a of the tube 21 is designed to be positioned in the anterior eye chamber between cornea and lens. The arrow P1 in FIG. 1 shows how the tube 21 of the implanted device 1 drains internal eye fluid (aqueous humor) from the anterior eye chamber (between the cornea and lens). By means of the tube 21 and the fluid flow channel 11 providing fluid communication between the fluid inlet 7 and the fluid outlet 9, the open reservoir 5 will be filled with aqueous humor. The overflow members 13, 13′, 13″, 13″′ distribute aqueous humor from the reservoir in a predetermined direction over the bulbus as indicated by arrows P2 in FIG. 1, i.e. posteriorly, such that bleb formation is directed further onto the bulbus, which facilitates optimal bleb formation for drainage by minimizing fibrosis of the bleb. The aqueous humor being pooled in the reservoir 5 and distributing the aqueous humor out of the device 1 by means of the overflow members 13, 13′, 13″, 13″′ provides optimal bleb formation around the implanted ocular drainage device 1 with minimal or no fibrosis such that an improved drainage of aqueous humor can be obtained by means of the device 1; 111; 211 of this disclosure.

As shown in the figures, the implantable ocular drainage device 1; 101; 201 has an open design provided by the at least upwards open reservoir and the endless wall body 3; 103; 203 provided with the overflow member 13, 13′, 13″, 13″′; 113, 113′, 113″; 213, 213′. Without being bound by theory, it is believed that this open-design of the implanted ocular drainage device contributes in providing an improved size and cell composition of the bleb around the implanted device. Optimal bleb formation around the implanted ocular drainage device 1; 101; 201 with minimal or no fibrosis improves drainage of aqueous humor from the eye in a natural manner. The device 1; 101; 201 is made of a made of a biocompatible, non-erodible polymer, preferably poly(styrene-block-isobutylene-block-styrene) (SIBS). SIBS causes minimal inflammation, scarring and capsule formation. The size and cell composition of the bleb to be formed around the device 1; 101; 201 are directly positively influenced by SIBS. Bleb control is improved by creating an entire glaucoma drainage device 1; 101; 201 from SIBS.

In the embodiments shown in the figures the wall body 3; 103; 203 is a ring-shaped wall body 3; 103; 203. The wall body may have a ring-like shape such as an ellipse-shaped wall body 3; 103; 203. A ring-or ellipse-shaped wall body have the advantage that these shapes are adapted to provide a stable and/or correct positioning on the eye-ball which contributes in a relatively high success rate of implanted devices 1; 101; 201. Alternatively, a wall body with another shape, for example rectangular or triangular, is also possible.

The maximum dimension of the wall body 3; 103; 203 defining the reservoir 5; 105; 205 is provided between outer portions of opposing body wall sections, see FIG. 5 indicated with D, wherein D is 15 mm or smaller, preferably 12 mm or smaller. The distance d between inner portions of opposing body wall sections is 8 mm or smaller, preferably 5 mm or smaller. The distance d defines the volume of the reservoir 5; 105; 205. The device 1; 101; 201 has relatively compact dimensions, i.e. the device 1; 101; 201 can be produced relatively small, which makes the device 1; 101; 201 easier to implant, but relatively effective in draining aqueous humor from the eye. Further, the height of the device 1; 101; 201 is relatively small compared to the maximum dimension of the wall body 3; 103; 203 and the height of the device is substantially provided by the height H1 of the body wall 3; 103; 203. The height H1 of the body wall 3; 103; 203 is larger than 0.5 mm and smaller than 3 mm.

In the device 1; 101; 201 shown in the figures, the wall body 3; 103; 203 comprises the at least one fluid inlet 7; 107; 207, the at least one fluid outlet 9; 109; 209 debouching into the reservoir 5; 105; 205 and the at least one fluid flow channel 11; 111; 211 providing fluid communication between the at least one fluid inlet 7; 107; 207 and the at least one fluid outlet 9; 109; 209. As shown in the figures, the at least one fluid flow channel 11; 111; 211 is located inside the wall body 3; 103; 203, wherein in a device 1; 201 with a single fluid outlet 9, the minimum length of the flow channel is 50% of the length of the endless wall body 3; 103. The minimum length of the flow channel to obtain a posteriorly located fluid outlet for filling the reservoir in the implanted device is at least 25% of the length of the endless wall body. In the embodiments shown, the flow channel 11; 111 has the same shape as the wall body 3; 103 and the ring-shaped flow channel 11; 111 extends like a ring inside the wall body such that the length of the flow channel 11; 111 corresponds substantially to the length of the endless wall body 3; 103.

As indicated with the dotted circle R in FIG. 5, it is possible to include the magnetic micro-valve mechanism as disclosed in WO2022/111892 in the ocular drainage device 1; 101; 201 of the present disclosure. For example, it is possible to combine the magnetic micro-valve mechanism as shown in FIGS. 5A-D of WO2022/111892 in the ocular drainage device 1; 101; 201 of the present disclosure, wherein the magnetic micro-valve mechanism is positioned in the reservoir volume, for example in the center as shown in FIG. 5 with the dotted circle. The reservoir is then mainly provided around the magnetic micro-valve mechanism (circle R) and the magnetic micro-valve mechanism is also surrounded by the endless wall body provided with the outflow member as disclosed herein. In such a configuration the at least one fluid outlet is provided by the magnetic micro-valve mechanism in its center. The fluid flow channel is extended in this configuration with the magnetic micro-valve mechanism for example by tube-like channels to provide fluid communication between the at least one fluid inlet 7; 107; 207 and the at least one fluid outlet provided in the known device, for example by tube-like channels (not shown) extending between the wall body at positions of the fluid outlets 109, 109′, 109″ shown FIG. 5 and housing channels of the magnetic micro-valve mechanism.

FIGS. 2 and 3 show more details of the device 1 shown in FIG. 1. With reference to FIG. 3, the height indicated with H1 between the top 3a of the wall body 3 and the bottom 3b of the wall body 3 provides the reservoir volume defined by means of the height H1 of the wall body 3, whereas the height H2 between the overflow member 13″ and the bottom 3b of the wall body provides the maximum volume of fluid in the reservoir 5 before fluid flows through the overflow member 13″ outside the device 1 such that the overflow members 13, 13′, 13″, 13″′ control the maximum volume of fluid in the reservoir 5. As will be clear from the height difference between H1 and H2 shown in FIG. 3 the maximum volume of fluid to be collected in the reservoir 5 is smaller than the reservoir volume. The same principle is also applied in the embodiments shown in FIGS. 4-6. In other words, the overflow members 13, 13′, 13″, 13″′; 113, 113′, 113″; 213, 213′ are notches in the top of the wall body 3; 103; 203, wherein the notches define the maximum volume of fluid to be collected in the reservoir 5; 105; 205. In principle, it is possible to use only one notch in the wall body of the device 1; 101; 201 to define the maximum volume, but to obtain a more optimal distribution of aqueous humor over/through the wall body 3; 103; 203 at least two notches in the wall body, preferably at least three notches in the wall body are provided.

As shown in figures, the endless wall body 3; 103; 203 of the device 1; 101; 201 has a center M (FIGS. 2 and 5, or point on center line C of wall body as shown in FIG. 4) providing a virtual origin used for defining a virtual x-(see for example cross section line in FIG. 2 indicated with X-X) and y-axis (dotted line y in FIG. 2), wherein, seen from above, the position of the fluid inlet 7 coincides with the y-axis and at least a portion of the at least one overflow member 13, 13′, 13″, 13″′; 113, 113′, 113″; 213, 213′ and/or at least a portion of the at least one fluid outlet 9; 109, 109′, 109″; 209 are provided in a wall body section of the wall body 3; 103; 203 opposite to the fluid inlet 7; 107; 207 with respect to the x-axis. In other words, the x-axis (see FIG. 2 dotted line X-X) divides the wall body 3; 103; 203 in two sections, wherein at least a portion of the at least one overflow member and/or at least a portion of the at least one fluid outlet is provided in the section of the two sections furthest away from the fluid inlet 7; 107; 207. Hence, the section of the two sections closest to the fluid inlet 7; 107; 207 cannot comprise an overflow member and/or a fluid outlet completely. In FIG. 2, it is demonstrated that each overflow member 13, 13″′ consists of two overflow member portions 13A, 13B, 13A′″, 13B′″, wherein only the portions 13A, 13A″′ are provided in the wall body section of the wall body 3 opposite to the fluid inlet 7 with respect to the x-axis. This configuration of the device 1; 101; 201 having at least one posteriorly located overflow member 13, 13′, 13″, 13″′; 113, 113′, 113″; 213, 213′, has the result that aqueous humor flow is distributed posteriorly, such that bleb formation is directed further onto the bulbus, which facilitates optimal bleb formation for drainage by minimizing fibrosis of the bleb. In addition, or alternatively, the posteriorly located fluid outlet(s) 9; 109, 109′, 109″; 209 for filling the reservoir in the implanted device maximizes tissue healing after implantation.

The implantable ocular device 101; 201 shown in FIGS. 4-6 differs from the implantable ocular device 1, in that the implantable ocular device 101; 201 is provided with a membrane 125; 225 connected to the wall body 103; 203 and providing a bottom of the reservoir 105; 205. The dimensions in the x-and y direction of the membrane 125; 225 are larger than the dimensions of the wall body 103; 203. The membrane 125; 225 has an oval shape seen from above (FIGS. 4 and 5). The maximum dimension of the membrane 125; 225 extending in the x-direction (FIG. 5) is 20 mm. The height of the membrane 125; 225 is smaller than the height of the body wall 103; 203. The height of the membrane 125; 225 is 0.5 mm or smaller, preferably approximately 0,1 mm. The membrane 125; 225 makes it possible to incorporate surface topography and/or openings 126 that allow control over the nearby cellular environment, for example promoting wound healing or tissue anchoring.

The device 101 further demonstrates that the at least one outlet may comprise at least two spaced outlet openings in the wall body 103, in particular three spaced outlet openings 109, 109′, 109″ in the wall body 103. These outlet openings 109, 109′, 109″ or at least a portion of each outlet openings 109, 109″ are provided in a wall body section of the wall body 103 opposite to the fluid inlet 107 with respect to the x-axis as explained above.

The device 1; 101 201 may comprise a curved-design adapted to the curvature of the eye. This curved-design of the device 201 is shown in FIG. 6. Such a curved-design facilitates positioning of the device in the eye during implantation and to retain its location after implantation, but also reduces strain and/or deformation after implanting the device which contributes in a more patient-friendly device after implantation.

Although not shown in the figures, it is also possible to provide an implantable ocular drainage device for controlling intraocular pressure, wherein the ocular drainage device comprises a wall body surrounding in an endless manner a reservoir with a reservoir volume defined by means of the wall body, wherein the wall body further comprises at least one fluid inlet, at least one fluid outlet for filling the reservoir with fluid and at least one internal fluid flow channel providing fluid communication between the at least one fluid inlet and the at least one fluid outlet, wherein the device has an open design provided by the at least upwards open reservoir. The ocular implantable drainage device with the reservoir defined by the wall body of the device designed to collect fluid (hereafter aqueous humor) in the reservoir, wherein after collecting a maximum volume of aqueous humor in the reservoir the aqueous humor leaves the device by overflow over the top of the wall body. Optimal bleb formation around the implanted ocular drainage device is achieved by the aqueous humor being pooled in the reservoir. The wall body in such a device may be ring-shaped or ellipse-shaped. The maximum dimension of the wall body is provided between outer portions of opposing body wall sections, wherein the maximum dimension is for example 15 mm, preferably 12 mm. The fluid outlet(s) and/or the (internal) fluid flow channel of such a device may be positioned as described above and as shown in the figures. In addition, such a device may comprise a curved-design adapted to the curvature of the eye as disclosed in this disclosure. The device may also be combined with a tube and/or a membrane connected to the wall body and providing a bottom of the reservoir as shown in the figures and disclosed above.

1. An implantable ocular drainage device for controlling intraocular pressure, wherein the ocular drainage device comprises a wall body surrounding in an endless manner a reservoir with a reservoir volume defined by means of the wall body, wherein the device further comprises at least one fluid inlet, at least one fluid outlet for filling the reservoir with fluid and at least one fluid flow channel providing fluid communication between the at least one fluid inlet and the at least one fluid outlet, wherein the wall body is provided with at least one overflow member configured to control a maximum volume of fluid in the reservoir, wherein the maximum volume of fluid in the reservoir is smaller than the reservoir volume.

2. The device according to claim 1, wherein, seen from above, the endless wall body defines a center providing a virtual origin used for defining a virtual x- and y-axis, wherein, seen from above, the position of the fluid inlet coincides with the y-axis and at least a portion of the at least one overflow member is provided in a wall body section of the wall body opposite to the fluid inlet with respect to the x-axis.

3. The device according to claim 1, wherein, seen from above, the endless wall body defines a center providing a virtual origin used for defining a virtual x-and y-axis, wherein, seen from above, the position of the fluid inlet coincides with the y-axis and at least a portion of the at least one fluid outlet is provided in a wall body section of the wall body opposite to the fluid inlet with respect to the x-axis.

4. The device according to claim 1, wherein the wall body is ring-shaped or ellipse-shaped.

5. The device according to claim 1, wherein the device has an open design provided by the at least upwards open reservoir and the endless wall body provided with the overflow member.

6. The device according to claim 1, wherein the at least one overflow member is a notch in the wall body, preferably the at least one overflow member comprises at least two notches in the wall body, more preferred at least three notches in the wall body.

7. The device according to claim 1, wherein the at least one outlet comprises at least two spaced outlet openings in the wall body, preferably at least three spaced outlet openings in the wall body.

8. The device according to claim 1, wherein the device further comprises a membrane connected to the wall body and providing a bottom of the reservoir.

9. The device according to claim 8, wherein the membrane incorporate surface topography and/or openings that allow control over the nearby cellular environment.

10. The device according to claim 1, wherein the wall body comprises the at least one fluid inlet, the at least one fluid outlet debouching into the reservoir and the at least one fluid flow channel providing fluid communication between the at least one fluid inlet and the at least one fluid outlet.

11. The device according to claim 1, wherein the device further comprises a tube, wherein the tube is connected or connectable to the fluid inlet.

12. The device according to claim 1, wherein the device comprises a curved-design adapted to the curvature of the eye.

13. The device according to claim 1, wherein the maximum dimension of the wall body is provided between outer portions of opposing body wall sections, wherein the maximum dimension is 15 mm, preferably 12 mm.

14. The device according to claim 1, wherein the at least one fluid flow channel is at least partly located inside the wall body with a length of at least 25% of the length of the endless wall body.

15. The device according to any claim 1, wherein the device is made of a made of a biocompatible, non-erodible polymer, preferably poly(styrene-block-isobutylene-block-styrene) and/or the device includes a magnetic micro-valve mechanism to control intraocular pressure in the eye.
